(19) United States
(12) Patent Application Publication
(10) Pub. No.: US 2010/0266590 A1
(43) Pub. Date: Oct. 21, 2010

(54) COMBINATION THERAPY

(76) Inventors: George D. Demetri, Brookline, MA (US); Stephen T. Sonis, Wayland, MA (US); Camille L. Bedrosian, Belmont, MA (US)

Correspondence Address: ARIAD PHARMACEUTICALS, INC.;ARIAD GENE THERAPEUTICS, INC., 26 LANDSDOWNE ST., CAMBRIDGE, MA, 02139, US

(21) Appl. No.: 12/309,665

(22) PCT Filed: Aug. 1, 2007
(86) PCT No.: PCT/US2007/017169

§ 371 Date: May 4, 2010

Related U.S. Application Data
(60) Provisional application No. 60/835,185, filed on Aug. 2, 2006.
(60) Provisional application No. 60/861,125, filed on Nov. 27, 2006.

(51) Int. Cl. A61K 39/395; A61P 35/00; A61P 35/02

(52) U.S. Cl. 424/134.1; 424/133.1; 424/142.1

(57) ABSTRACT

Disclosed are methods for treating various cancers. Methods encompass the administration of a first drug such as AP23573, temsirolimus or everolimus in combination with a second drug selected from Remicade, Humira, Enbrel, Raptiva, Abatacept, Actermra, Cimzia or anakinra.

The methods are aimed at providing a desirable therapeutic window while maintaining prior, if not higher, dose levels of the first drug.

### BACKGROUND

Several mTOR inhibitors are currently under evaluation as single agents or in various combinations for the treatment of a variety of cancers. Those mTOR inhibitors include the rapamycin analogs, AP23573 (ARIAD Pharmaceuticals, Inc.), everolimus (Novartis) and temsirolimus (Wyeth). Other mTOR inhibitors include, among others, sirolimus (rapamycin), and the additional analogs, ABT-578 and biolimus. While AP23573, everolimus and temsirolimus have all yielded positive results in human studies, mouth sores have been noted as a dose limiting toxicity.

Those mouth sores have previously been loosely termed “mucositis” in some cases. Actually, however, they typically differ noticeably from the classic mucositis that frequently accompanies radiation therapy and other cancer therapies such as cytotoxic cancer chemotherapies. Nonetheless, these mouth sores can be debilitating and constitute a dose limiting toxicity for the use of the new mTOR inhibitors.

One approach sometimes used in order to reduce drug doses and thus to alleviate side effects is to shift from monotherapy to combination therapy, where the addition of a second drug permits smaller doses of the first drug, and thus, reduced incidence or severity of side effects. This invention takes a somewhat different approach.

### SUMMARY OF THE INVENTION

We now provide a combination therapy for achieving a desirable therapeutic window with a rapamycin analog while maintaining prior, if not higher, dose levels. The method involves administering to a cancer patient a rapamycin analog in combination with the administration of a second drug selected from Remicade, Humira, Enbrel, Raptiva, Orencia, Gollmumab, Actemra, Cimzia and Kineret.

The rapamycin analog is selected from AP23573, temsirolimus and everolimus, and may be administered by any of the known, or otherwise pharmaceutically acceptable, routes for that class of drugs. Oral or parenteral administration is currently of particular interest.

Therapeutically effective (adult) dose levels for the rapamycin analog in this combination therapy are in the range of 25-800 mg/week. In some cases it is in the range of 100-500 mg/week. The weekly dosage level may be achieved for example with average daily oral administration of 5-160 mg provided up to 7 days per week (e.g., 30-100 mg QDx6; i.e. administration of 30-100 mg/day for 6 consecutive days), or with i.v. infusions one or more times per 7-21-day period. Of particular interest are dose levels and schedules at least as high/frequent as those approved or under study for monotherapy and/or for combination with drugs other than the “second drugs” of this document. In dosing schedules involving administration of the rapamycin analog for 3 or more days per week, e.g., every day, QDx4, QDx5, or QDx6 (i.e administration of the rapamycin analog for 4, 5 or 6 consecutive days), doses of 20 mg and greater are of special interest, and are of increasing interest at or above levels of 30 mg, 40 mg, 50 mg, and 60 mg and higher. The dosing schedule may be intermittent. “Intermittent” dosing refers to schedules providing intervening periods between doses, e.g. every second day dosing, every third day dosing, or more generally, schedules containing “holidays” of one or more days or weeks between periods of dosing. A non-limiting example of such intermittent dosing is a cycle of one week of QDx4 (or QDx5 or QDx6) dosing followed by one week (or two or three weeks) without drug, then resuming with another week of drug treatment followed by a week (or weeks) without drug treatment, and so on. To illustrate further, administration of 60 mg QDx6 every other week provides a weekly dose of 360 mg of drug on an intermittent basis (i.e., every other week).

Each of the specified second drugs has been approved or studied at a range of acceptable dose levels which shall be referred to as “therapeutically effective” dose levels for the purpose of this document. Of particular interest are doses approved for monotherapy or other combination therapies involving administration of the respective second drug, as well as doses effective to reduce the incidence or severity of mouth sores which can accompany administration of an mTOR Inhibitor. In the practice of the method described herein, the second drug is administered by any route useful for such second drugs, usually by intravenous or subcutaneous administration.

Therapeutically effective amounts of one of the rapamycin analogs and one of the second drugs, respectively, are co-administered to the patient, i.e., are administered to the patient simultaneously or sequentially, in any given order and by the same or different routes of administration. It is of particular current interest to initiate administration of the second drug first, for example, one or more days or weeks prior to initiation of administration of the rapamycin analog.

In some cases the rapamycin analog is given orally and the second drug is administered intravenously, subcutaneously or topically. In other cases the rapamycin analog is given by intravenous infusion and the second drug is administered, as above, intravenously, subcutaneously or topically.

Given the documented activity of the rapamycin analogs against a wide variety of cancers, the combination therapy disclosed herein should be of interest for a correspondingly wide range of cancers. Those include among others prostate, endometrial, breast, ovarian, cervical, head and neck, small cell and non-small cell lung, pancreatic, kidney, brain, colorectal and bladder cancers as well as various sarcomas (including the various bone and soft tissue sarcomas), melanomas, multiple myeloma, B-cell lymphoma, mantle cell lymphoma, Non-Hodgkin's Lymphoma, CLL and CML, including, among others, cases which are advanced, recurrent, refractory to one or more other therapies and/or metastatic.

Moreover, additional drugs such as those described further below may be given in conjunction with the combination therapy of this invention.

Another aspect of this invention is a method for enhancing the efficacy, and/or decreasing the incidence and/or severity of side effects (e.g., mouth sores), associated with administration to a cancer patient of an mTOR inhibitor such as one of the specified rapamycin analogs, the method involving administration to the same patient one of the specified second drugs, in an amount effective for such purpose, before, during or after initiation of treatment with the mTOR inhibitor.

### DETAILED DESCRIPTION

The combination therapy disclosed herein thus constitutes a new method for treating various types of cancer, a method providing a desirable therapeutic window for achieving clinical benefit without incurring an unacceptable level of side effects.

As used herein, the term “treating” refers to the co-administration of an mTOR inhibitor and a second drug to a patient after the onset, or suspected onset, of a cancer. “Treating” includes the concepts of “alleviating”, which refers to lessening the frequency of occurrence or recurrence, or the severity, of any symptoms or other ill effects related to a cancer and/or the side effects associated with cancer therapy. The term “treating” also encompasses the concept of “managing” which refers to reducing the severity of a particular disease or disorder in a patient or delaying its recurrence, e.g., lengthening the period of remission in a patient who had suffered from the disease.

The term “effective amount” or “effective dose”, as used herein means the amount or dose effective for preventing, treating, or lessening the severity of symptoms (e.g. symptoms related to various types of cancers, or symptoms related to side effects of anti cancer therapy).

Numerical ranges in this document are intended to include every number and subset of numbers contained within that range, whether specifically disclosed or not. Moreover, these numerical ranges are intended to cover all included numbers and ranges within the recited range. For example, a disclosure of from 1 to 10 is intended to include a range of from 2 to 8, from 5 to 6, from 4.2 to 6.6, from 4.1 to 9.9, etc.

All references to singular characteristics or limitations are intended to include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination of steps is made.

All percentages, parts and ratios as used herein are by weight of the total composition, unless otherwise specified. All such weights as they pertain to listed ingredients are based on each respective ingredient per se and, therefore; do not include solvents or by-products that may be included in commercially available materials, unless otherwise specified.

The compositions and methods of the present invention can comprise additional or optional ingredients, components, or limitations described herein or otherwise useful in compositions and methods of the general type as described herein.

1. Rapamycin Analogs—mTOR Inhibitors

Rapamycin is a macrolide produced by Streptomyces hygroscopicus and discovered in the 1970's. Rapamycin is a potent immunosuppressive agent and is used clinically to prevent rejection of transplanted organs. It has also been reported to have a wide range of interesting pharmacologic activities, including certain anti-cancer activity. See e.g. US Pat. appln 2001/0010920. A number of derivatives of rapamycin, including AP23573 (ARIAD), CCI779 (“temsirolimus”, Wyeth) and RAD001 (“Everolimus”, Novartis) are currently in clinical development as anti-cancer agents and have yielded promising results in human studies against a variety of cancers. In addition, rapamycin and everolimus are used as immunosuppressants in organ transplant recipients. Rapamycin and a number of the C-43-modified rapamycin analogs, including among others AP23573, Biolimus and ABT-578 (Abbott), are being used, evaluated or developed for use on drug-eluting stents.

Because there is more than one accepted convention for numbering the positions of rapamycin, and derivatives thereof, the numbering convention used herein is depicted below: For reference, the R group for a number of compounds is set forth in the following table:

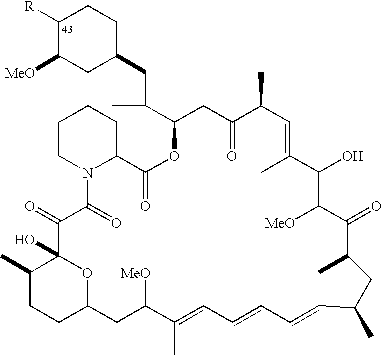

| R | Compound |
|---|---|
| HO— | rapamycin |
| (Me)₂P(O)—O— | AP23573 |
| (HOCH₂)₂C(CH₃)CO₂— | temsirolimus |
| HOCH₂CH₂O— | everolimus |

Temsirolimus (CCI-779) is disclosed in U.S. Pat. No. 5,362,718. CCI-779 has demonstrated significant inhibitory effects on tumor growth in both in vitro and in vivo models. CCI-779 may delay the time to progression of tumors or time to tumor recurrence. As disclosed in WO 00/240000, CCI-779 may be useful for the treatment of cancers of various origins, including renal, breast, cervical, uterine, head and neck, lung, prostate, pancreatic, ovarian, colon, lymphoma and melanoma. For additional references on Temsirolimus, see WO 2004/026280, WO 2005/070393, WO 2006/086172 and WO 2006/08932.

Everolimus “RAD001”, “SDZ RAD”, Certican™) is disclosed by structure and synthesis of which in WO 94/09010. RAD001, which has been shown to be a potent immunosuppressive agent (U.S. Pat. No. 5,665,772), also exhibits antineoplastic properties (see, e.g., A. Boulay of at, Cancer Res. 2004, 64: 252-261). RAD001 is currently marketed in certain countries as an immunosuppressant for prevention of allograft rejection (B. Nashan, Ther. Drug. Mona., 2002, 24: 53-58) and is undergoing clinical studies as an anti-cancer agent (S. Huang and P. J. Hougthon, Curr. Opin. Invest. Drugs, 2002, 3: 295-304; M. M. Mita et al., Clin. Breast Cancer, 2003, 4: 126-137: M. Hidalgo and E. J. Rowinsky, Oncogene, 2000, 19: 6680-6686). For additional references on Everolimus, see U.S. Pat. No. 6,384,046, U.S. Pat. No. 6,197,781, U.S. Pat. No. 6,004,973, WO 2002/066019 and references cited therein.

The analog of special interest is AP23573, a phosphorous-containing rapamycin derivative disclosed in WO 03/064383 and U.S. Pat. No. 7,091,213. Like CCl-779 and RAD001, AP23573 has demonstrated antiproliferative activity in a variety tumor dell lines, including glioblastoma, prostate, breast, pancreas, lung and colon (E. K. Rowinsky, Curr. Opin. Oncol., 2004, 16: 564-575). AP23573 has recently been designated as a fast-track product by the U.S. Food and Drug Administration for the treatment of soft-tissue and bone sarcomas. AP23573 is currently in multiple Phase 2 and 1b clinical trials as a single agent in patients with hematologic malignancies (e.g., leukemias and lymphomas) and solid tumors (e.g., sarcomas, prostate cancer and gliobastoma multiforme). Studies of AP23573 in combination with other drugs are also underway.

2. Second Drug

The second drug of the combination therapy is selected from Remicade (Infliximab), Humira (adalimumab), Enbrel (etanercept), Raptiva (efalizumab), Orencia (abatacept), Golimumab, Actemra (tocilizumab), Cimzia (certolizumab) and Kineret (anakinra), and may be administered using the same product form, dose level, dose frequency and route of administration used for other applications of that second drug. While such information is well known to the practitioner, the following brief review may be useful to some readers.

Remicade^{R} (infliximab) is a chimeric monoclonal antibody (the term “chimeric” refers to the use of both mouse and human components of the drug i.e. mouse binding VK and VH domains and human constant Fc domains).

Infliximab is approved for use alone or combined with methotrexate for treating moderate to severe rheumatoid arthritis. Infliximab is used for reducing the signs and symptoms of Inflammation in moderate to severe Crohn's disease. It also is approved for the treatment of active psoriatic arthritis. Infliximab has been reported to be helpful in reducing the joint inflammation of juvenile rheumatoid arthritis, ankylosing spondylitis, uveitis, psoriasis, and for sarcoidosis that is not responding to traditional therapies. Remicade is also sometimes used to treat ulcerative colitis (inflammation and sores in the large intestine), skin diseases, juvenile arthritis (joint pain and swelling in children), and Behcet's syndrome (ulcers in the mouth and on the genitals and inflammation of various parts of the body).

Infliximab comes as a powder to be mixed with sterile water and infused intravenously. More specifically, the powder is reconstituted by dilution in 10 mL of Sterile water. The total dose of reconstituted product is then further diluted to 250 mL with 0.9% Sodium Chloride for injection. It is not suitable for oral administration, because it would decompose in the digestive system prior to entering the blood stream.

The recommended dose is 3 to 5 mg/kg as a single dose. The initial dose should be followed by an additional 3 to 5 mg/kg doses two and six weeks after the first dose. Thereafter, the maintenance dose is 3 mg/kg every eight weeks.

Enbrel (Etanercept) is a small fusion protein, with a molecular weight of 75 kDa, containing two naturally occurring soluble human 75-kilodalton TNF receptors linked to an Fc portion of an IgG1.

Enbrel is indicated for reducing the signs and symptoms of rheumatoid arthritis, ankylosing spondylitis, psoriatic arthritis, and chronic moderate to severe psoriasis. It is also currently undergoing clinical trials for the treatment of stomatitis and oral pain in patients receiving chemotherapy.

Enbrel is marketed as a powder which must be reconstituted with a diluent and then injected subcutaneously, typically by the patient at home. It also is not suitable for oral administration.

The recommended dose of Enbrel for adult patients with rheumatoid arthritis, psoriatic arthritis, or ankylosing spondylitis is 50 mg per week given as one subcutaneous injection using a 50 mg/mL single-use pre-filled syringe.

The recommended starting dose of Enbrel for adult patients is a 50 mg dose given twice weekly (administered 3 or 4 days apart) for 3 months followed by a reduction to a maintenance dose of 50 mg per week.

While infliximab is a mouse-human chimeric antibody and etanercept is an IgG fusion protein, Humira (adalimumab) is a fully human monoclonal antibody. Humira® is approved for reducing the signs and symptoms, inducing major clinical response, slowing the progression of joint damage, and Improving physical function in adult patients with moderate to severe rheumatoid arthritis. Humira is also approved for reducing the signs and symptoms of active arthritis in patients with psoriatic arthritis.

Humira is marketed in preloaded 0.8 ml syringes and is injected subcutaneously, typically by the patient at home. The recommended dose of Humira for adult patients with rheumatoid arthritis or psoriatic arthritis is 40 mg administered every other week as a subcutaneous injection. In rheumatoid arthritis, some patients may derive additional benefit from increasing the dosing frequency of Humira to 40 mg every week.

Efalizumab (Raptiva®, Genentech) is a humanized monoclonal antibody that binds to CD11a and is approved as a once a week subcutaneous Injection for the treatment of psoriasis. It is provided as a vial of 125 mg lyophilized (freeze-dried) powder, with a pre-filled syringe containing sterile water for reconstitution, and syringes and needles for injection. The first dose is 0.7 mg per kilogram of body weight. Thereafter, each weekly dose is 1 mg per kilogram (up to a maximum single dose of 200 mg).

Abatacept (Orencia) is a human fusion protein categorized as a costimulatory or second-signal blocker of T cell activation. Abatacept is thought to disrupt the activation pathway of T cells causing a disturbance in key mechanisms of inflammation and progressive joint destruction in rheumatoid arthritis. ORENCIA should be administered as a 30-minute intravenous infusion at the dose specified in the Table below. Following the initial administration, ORENCIA should be given at 2 and 4 weeks after the first infusion, then every 4 weeks thereafter. ORENCIA may be used as monotherapy or concomitantly with disease-modifying, anti-rheumatic drugs (DMARDs) other than TNF antagonists.

TABLE
Dose of ORENCIA

| | | |
|---|---|---|
| Body Weight of Patient | Dose | Number of Vials^{a} |

| | | | |
|---|---|---|---|
| <60 kg | 500 | mg | 2 |
| 60 to 100 kg | 750 | mg | 3 |
| >100 kg | 1 | gram | 4 |

Golimumab (Centocor and Schering-Plough) is a fully-human anti-TNF-alpha IgG1 monoclonal antibody that targets and neutralizes both the soluble and the membrane-bound form of TNF-alpha. Golimumab is currently being investigated as a subcutaneous (SC) injection or intravenous (IV) infusion for the treatment of active psoriatic arthritis, rheumatoid arthritis and active ankylosing spondylitis, administered as a 50 mg-100 mg injection every 2 or 4 weeks.

ACTEMRA® (Tocilizumab) is a humanized monoclonal antibody (Osaka University and Chugai) approved for treating Castleman's disease and under investigation for the treatment of rheumatoid arthritis.

Dosage and Administration: Tocilizumab is administered as an 8 mg/kg intravenous infusion administered every 2 or 4 weeks. The dosing interval may be shortened to 1 week as minimum depending on the patient's condition.

Cimzia™: certolizumab pegol (UCB) is a PEGylated Fab′ fragment of a humanized anti-TNF alpha antibody in development for treating rheumatoid arthritis and Crohn's disease, administered as a 400 mg injection every four weeks (after a two week induction phase administration).

Kineret® (anakinra) is a recombinant, nonglycosylated form of the human interleukin-1 receptor antagonist (IL-1Ra). It is supplied as a treatment for rheumatoid arthritis in single-use preservative free, prefilled glass syringes with 27 gauge needles. Each pre-filled glass syringe contains 0.67 mL (100 mg) of anakinra.

3. Indications

Examples of cancers and cancer conditions that can be treated with the combination therapy of this document include, but are not limited to, tumors of the brain and central nervous system (e.g., tumors of the meninges, brain, spinal cord, cranial nerves and other parts of the CNS, such as glioblastomas or medulla blastomas); head and/or neck cancer, breast tumors, tumors of the circulatory system (e.g., heart, mediastinum and pleura, and other intrathoracic organs, vascular tumors, and tumor-associated vascular tissue); tumors of the blood and lymphatic system (e.g., Hodgkin's disease, Non-Hodgkin's disease lymphoma, Burkitt's lymphoma, AIDS-related lymphomas, malignant immunoproliferative diseases, multiple myeloma, and malignant plasma cell neoplasms, lymphoid leukemia, myeloid leukemia, acute or chronic lymphocytic leukemia, monocytic leukemia, other leukemias of specific cell type, leukemia of unspecified cell type, unspecified malignant neoplasms of lymphoid, hematopoietic and related tissues, such as diffuse large cell lymphoma, T-cell lymphoma or cutaneous T-cell lymphoma); tumors of the excretory system (e.g., kidney, renal pelvis, ureter, bladder, and other urinary organs); tumors of the gastronintestinal tract (e.g., esophagus, stomach, small intestine, colon, colorectal, rectosigmoid junction, rectum, anus, and anal canal); tumors involving the liver and intrahepatic bile ducts, gall bladder, and other parts of the biliary tract, pancreas, and other digestive organs; tumors of the oral cavity (e.g., lip, tongue, gum, floor of mouth, palate, parotid gland, salivary glands, tonsil, oropharynx, nasopharynx, puriform sinus, hypopharynx, and other sites of the oral cavity); tumors of the reproductive system (e.g., vulva, vagina, Cervix uteri, uterus, ovary, and other sites associated with female genital organs, placenta, penis, prostate, testis, and other sites associated with male genital organs); tumors of the respiratory tract (e.g., nasal cavity, middle ear, accessory sinuses, larynx, trachea, bronchus and lung, such as small cell lung cancer and non-small cell lung cancer); tumors of the skeletal system (e.g., bone and articular cartilage of limbs, bone articular cartilage and other sites); tumors of the skin (e.g., malignant melanoma of the skin, non-melanoma skin cancer, basal cell carcinoma of skin, squamous cell carcinoma of skin, mesothelioma, Kaposi's sarcoma); and tumors involving other tissues including peripheral nerves and autonomic nervous system, connective and soft tissue, retroperitoneoum and peritoneum, eye and adnexa, thyroid, adrenal gland, and other endocrine glands and related structures, secondary and unspecified malignant neoplasms of lymph nodes, secondary malignant neoplasm of respiratory and digestive systems and secondary malignant neoplasm of other sites.

Of special interest is the treatment of sarcomas, bladder cancer, breast cancer, chronic lymphoma leukemia, head and neck cancer, endometrial cancer, Non-Hodgkin's lymphoma, non-small cell lung cancer, ovarian cancer, pancreatic cancer and prostate cancer.

Cancers that can be treated using this combination therapy include among others cases which are refractory to treatment with other chemotherapeutics. The term “refractory”, as used herein refers to a cancer (and/or metastases thereof), which shows no or only weak anti-proliferative response (e.g., no or only weak inhibition of tumor growth) after treatment with another chemotherapeutic agent. These are cancers that cannot be treated satisfactorily with other chemotherapeutics. Refractory cancers encompass not only (i) cancers where one or more chemotherapeutics have already failed during treatment of a patient, but also (ii) cancers that can be shown to be refractory by other means, e.g., biopsy and culture in the presence of chemotherapeutics.

The combination therapy described herein is also applicable to the treatment of patients who have not been previously treated.

Also of special interest is the treatment of PTEN-deficient cancers (see, for example, M. S. Neshat et al., PNAS, 2001, 98: 10314-10319; K. Podsypanina et al., PNAS, 2001, 98: 101320-10325; G. B. Mills et al., PNAS, 2001, 98: 10031-10033; M. Hidalgo and E. K. Rowinski, Oncogene, 2000, 19: 6680-6686). PTEN-deficient tumors may be identified, using genotype analysis and/or in vitro culture and study of biopsied tumor samples. Non-limiting examples of cancers involving abnormalities in the phosphatidyl-inositol 3 kinase/Akt-mTOR pathway include, but are not limited to, glioma, lymphoma and tumors of the lung, bladder, ovary, endometrium, prostate or cervix which are associated with abnormal growth factor receptors (e.g., EGFR, PDGFR, IGF-R and IL-2); ovarian tumors which are associated with abnormalities in P13 kinase; melanoma and tumors of the breast, prostate or endometrium which are associated with abnormalities in PTEN; breast, gastric, ovarian, pancreatic, and prostate cancers associated with abnormalities with Akt; lymphoma, cancers of the breast or bladder and head and neck carcinoma associated with abnormalities in elF-4E; mantle cell lymphoma, breast cancer and head and neck carcinomas associated with abnormalities in Cyclin D; and familial melanoma and pancreas carcinomas associated with abnormalities in P16.

4. Formulations, Administration and Use

Formulation of the mTOR Inhibitor:

A variety of oral and parenteral dosage forms are known for rapamycin and a number of rapamycin analogs. Some are currently in use in various treatment methods, monotherapies or otherwise. Those same dosage forms may likewise be used in the practice of the combination therapy disclosed herein. Solid dosage forms are often preferred for oral administration and include among others conventional admixtures, solid dispersions and nanopartides, typically in tablet, capsule, caplet, gel cap or other solid or partially solid form. Such formulations may optionally contain an enteric coating. Numerous materials and methods for such oral formulations are well known. A typical example of the use of conventional materials and methods to formulate an mTOR inhibitor is shown in US Patent Application US 2004/0077677 and Published International Patent Application WO04026280 (CCl-779). See also U.S. Pat. No. 6,197,781, U.S. Pat. No. 6,589,536, U.S. Pat. No. 6,555,132, U.S. Pat. No. 5,985,321, U.S. Pat. No. 6,565,859 and U.S. Pat. No. 5,932,243. In addition to the foregoing nonlimiting examples of formulation technology, a wide variety of other methods and materials are also well known to those working in the field of macrolides like rapamycin and its derivatives. For additional background and examples of appropriate formulation technologies, see e.g., WO 03/064383 and US Published Patent Application 20050032825.

In a preferred embodiment, the mTOR inhibitor is provided as an oral dosage form, such as a tablet. In the case of AP23573, for Instance, the drug may prepared by a wet granulation process. The tablet may contain one or more cellulose polymers and one or more of an antioxidant, chelating agent, filler, binder, surfactant, desintegrant, lubricant, pH-modifying agent and the like. The wet granulation process may be performed with an aqueous or alcoholic, e.g., ethanol, solvent system. Other suitable alcohols include methanol, isopropanol, and the like. The solvent can also be a mixture of solvents, e.g. an alcoholic solvent and water.

It is currently of particular interest that the composition contain from 1 to 45%, from 2 to 35%, from 5 to 25%, or from 8 to 15% by weight of AP23573; from 1 to 50%, from 1 to 35%, from 1 to 15%, or from 2 to 15% by weight of cellulose polymer and from 0.01% to 3%, from 0.05% to 1% or from 0.05% to 0.5% by weight of antioxidant. However, various embodiments may contain more, or less, of these components.

Acceptable antioxidants include, but are not limited to, citric acid, d,l-α-tocopherol, BHA, BHT, monothioglycerol, ascorbic acid, and propyl gallate. It is expected that the antioxidants of the formulations of this invention will be used in concentrations ranging from 0.001% to 3% wt/wt.

Chelating agents, and other materials capable of binding metal ions, such as ethylene diamine tetra acetic acid (EDTA) and Its salts are capable of enhancing the stability of AP23573.

Typical cellulose polymers include, but are not limited to hydroxypropylmethylcellulose (HPMC), hydroxypropylmethyl cellulose phthalate, methyl cellulose (MC), hydroxyethyl cellulose, and hydroxypropyl cellulose (HPC).

Acceptable pH modifying agents include, but are not limited to citric acid, sodium citrate, dilute HCl, and other mild acids or bases capable of buffering a solution containing AP23573 to a pH in the range of about 4 to about 6. If present in the composition, the pH modifying agent is usually in amount of up to 1%.

Surfactants may be present in the formulation and include polysorbate 80, sodium lauryl sulfate, sodium dodecyl sulfate, salts of bile acids (taurocholate, glycocholate, cholate, deoxycholate, etc.) which may be combined with lecithin. Alternatively, ethoxylated vegetable oils, such as Cremophor EL, vitamin E tocopherol propylene glycol succinate (Vitamin E TGPS), polyoxyethylene-polyoxypropytene block copolymers, and poloxamers. If present in the composition, the surfactant is usually in amount of up to 20%, for example 1 to 15% by weight.

Binders, fillers, and disintegrants such as sucrose, lactose, microcrystalline cellulose, croscarmellose sodium, magnesium stearate, gum acacia, cholesterol, tragacanth, stearic acid, gelatin, casein, lecithin (phosphatides), carboxymethylcellulose calcium, carboxymethylcellulose sodium, methylcellulose, hydroxyethylcellulose, hydroxypropylcellulose, hydroxypropylmethyl-cellulose phthalate, noncrystalline cellulose, polyvinylpyrrolidone, cetostearyl alcohol, cetyl alcohol, cetyl esters wax, dextrates, dextrin, cyclodextrin, lactose, dextrose, glyceryl monooleate, glyceryl monostearate, glyceryl palmitostearate, polyoxyethylene alkyl ethers, polyethylene glycols, polyoxyethylene castor oil derivatives, polyoxyethylene stearates, and polyvinyl alcohol, and the like may also be incorporated into the formulation.

Any given formulation of this invention may contain multiple ingredients of each class of component. For example, a formulation containing an antioxidant may contain one or more antioxidants as the antioxidant component.

The tablet may further comprise a film-coat to control the release of the rapamycin analog. The tablet may be coated with a film-coat by spraying, dipping or by deposition. The film-coat typically includes a polymeric film-forming material such as copovidone (i.e a copolymer of polyvinylpyrrolidone and vinyl acetate), hydroxypropyl methylcellulose, hydroxypropylcellulose, and acrylate or methacrylate copolymers. Besides a film-forming polymer, the film-coat may further comprise a plasticizer, e.g. polyethylene glycol, triethyl citrate, a surfactant, e.g. a Tween® type, an anti-foaming agent, e.g. Simethicone, and optionally a pigment, e.g. titanium dioxide or iron oxides. The film-coating may also comprise talc as anti-adhesive. The film coat usually accounts for less than about 5% by weight of the dosage form. In a preferred embodiment, the film-coating material comprises copovidone.

The film coating may also be an enteric layer comprising an enteric polymer, for delayed release of the rapamycin analog. An enteric layer is a coating of a substance (i.e a polymer) which is insoluble in the acid medium of the stomach but which is soluble at the higher pH encountered in the intestine. Such materials are used as film coatings on tablets to modify the release of a drug. Suitable enteric polymers are well known to those of skill in the art (WO 01/051031) and include, without limitation, methyl metacrylate polymers, methacrylic acid co-polymers, cellulose acetate phthalate, polyvinyacetate phthalate, hydroxypropyl methyl phthalate, and hydroxypropyl methyl cellulose phthalate. For instance, the enteric layer may comprise a methacrylic acid co-polymer such as Eudragit L100, Acryl-EZE or the like.

In addition to the foregoing non limiting examples of formulation technology, a wide variety of other methods and materials are also well known to those working in the field of macrolides like rapamycin and its derivatives. For additional background and examples of appropriate formulation technologies, see e.g., WO 03/064383 and US Published Patent Application 20050032825.

### Formulation of the “Second” Drug:

The second drug of the present invention can be administered by any routes; parenteral administration, i.e., intravenous, subcutaneous, intramuscular, is ordinarily used to optimize absorption.

For parenteral administration, the second drug of the invention can be formulated as a solution, suspension, emulsion or lyophilized powder in association with a pharmaceutically acceptable parenteral vehicle. Examples of such vehicles are water, saline, Ringer's solution, dextrose solution, and 5% human serum albumin. Liposomes and nonaqueous vehicles such as fixed oils can also be used. The vehicle or lyophilized powder can contain additives that maintain isotonicity (e.g., sodium chloride, mannitol) and chemical stability (e.g., buffers and preservatives). The formulation is sterilized by commonly used techniques.

In addition to the most commonly used formulations for parental administration of the second drug, other pharmaceutical acceptable carriers can be used. Suitable pharmaceutical carriers for preparing sterile solutions of the second drug are well known in the art and are described in Remington's Pharmaceutical Sciences, A. Osol, a standard reference text in this field of art.

The composition containing the second drug for use in the disclosed method, can also be prepared in any known or otherwise effective form suitable for use in providing topical or local delivery of the second drug to the affected mucosa.

In certain embodiments, the composition is administered orally in a form permitting it to contact and in some cases to coat, preferably rapidly, the oral and/or esophageal mucosa, to thus provide more effective contact with the affected mucosal tissue. Suitable formulations for topical administration to oral mucosa include liquid formulations (e.g., for mouth rinse, gargle, swish, mouthwash, spray, etc.), solid dosage forms which dissolve in the mouth, and semisolid dosage forms which are applied to coat oral surfaces. Dosage or product forms of this sort include mouthwashes which the individual may swish and swallow or swish and spit out. Suitable dosage forms also include oral lozenges, tablets, gels, and other forms described herein.

Compositions and methods of the present invention can be practiced with any pharmaceutically acceptable product form, usually liquid, that can directly or indirectly affect those areas of mucosa which have become or will likely develop one or more lesions following administration of an mTOR inhibitor.

Suitable compositions may be prepared by any known or otherwise effective method for formulating or manufacturing the selected product form. For example, the second drug can be formulated along with common excipients, diluents, or carriers, and formed into oral tablets, capsules, sprays, mouth washes, mouth rinse, swishes, lozenges, treated substrates (e.g., oral or topical swabs, pads, or disposable, non-digestible substrate treated with the compositions of the present invention); oral liquids (e.g., suspensions, solutions, emulsions), powders, or any other suitable dosage form for topical administration to oral mucosa.

In certain embodiments, the second drug is formulated as an elixir, solution, emulsion or suspension for convenient topical oral administration.

Such compositions further include pharmaceutical dosage forms such as lozenges, troches, lollipop, or pastilles. These are typically discoid or otherwise shaped solids containing the active ingredient in a suitably flavored base. The base may be a hard sugar or sugar-free candy, glycerinated gelatin, or the combination of sugar with sufficient mucilage to give it form. Troches are placed in the mouth where they slowly dissolve, liberating the active ingredient for direct contact with the affected mucosa.

Troche embodiments are prepared, for example, by adding water slowly to a mixture of the powdered active, powdered sugar, and a gum until a pliable mass is formed. A 7% acacia powder can be used to provide sufficient adhesiveness to the mass. The mass is rolled out and the troche pieces cut from the flattened mass, or the mass can be rolled into a cylinder and divided. Each cut or divided piece is shaped and allowed to dry, to thus form the troche dosage form.

When the second drug is heat stable, or can be rendered heat stable by the use of appropriate processing precautions, it may be prepared in the form of a hard candy base. For example, sugar containing syrup can be concentrated to the point where it becomes a pliable mass. The active ingredient is then added to the mass, which is then kneaded while warm to form a homogeneous mass.

The homogeneous mass is gradually worked into a pipe form having the diameter desired for the candy piece. Lozenges can be cut or sectioned off from the pipe and allowed to cool.

When the second drug is heat labile, it may be made into a lozenge preparation by compression. For example, the granulation step in the preparation is performed in a manner similar to that used for any compressed tablet. The lozenge is made using heavy compression equipment to give a tablet that is harder than usual as it is desirable for the dosage form to dissolve or disintegrate slowly in the mouth. Ingredients are preferably selected to promote slow-dissolving characteristics.

Alternative manufacturing approaches may, of course, be used for any of the various formulations.

In certain embodiments, the second drug is provided in a form which is topically administered to the oral mucosa and then either swallowed or spit out. Formulation types suitable for this route of administration include liquids applied as mouth rinses; solid dosage forms that may dissolve in the mouth; and semisolids that may be applied to oral cavity surfaces.

Stability of the various second drugs varies with structure. However, solids for re-constitution as aqueous based solutions or suspensions prepared either by the patient or by a pharmacist prior to administration to the patient can be used, even for the less stable members.

In some cases the stability of a compound in aqueous solution is pH dependent. Procedures for choosing the optimum pH and buffering agents are well known. Other factors that affect stability in solution are also well known. For example, antioxidants may be added to reduce the rate of degradation due to oxidation.

While the second drug may be in solution or suspension in a simple aqueous system, e.g., in a solution of sodium bicarbonate, the liquid preparation may further contain additional ingredients such as buffers (as mentioned above), surfactants, humectants, preservatives, flavorings, stabilizers (including antioxidants), colorants, and other additives used in preparations administered into the oral cavity. For example, the second drug may be stirred into a glass of water to which a dose of AlkaSeltzer is added and the mixture (solution or suspension) is swished and spit out or swished and swallowed. AlkaSeltzer is a branded, rapidly dissolving formulation of aspirin, citric acid and sodium bicarbonate used to treat headache, indigestion and hangover.

In certain embodiments, compositions used as mouthwashes have a pH of about 3.5 to about 8. A pH in the range of 4 to 6.5 is also contemplated. Compositions having a pH below 3.5 to 4 can cause a stinging sensation and preparations above pH 6.5 to 8 can be unpleasant to use. However, such unpleasant characteristics can be masked or otherwise made less troublesome for administration using conventional materials and methods.

The preparations are buffered as necessary to provide the appropriate pH. Appropriate buffer systems can include, among others, citrate, acetate, tromethamine and benzoate systems. However, any buffer system commonly used for preparing medicinal compositions would be appropriate. Suitable vehicles include water, alcohols, glycols (polyethylene glycol or polypropylene glycol are examples), glycerin, and the like which are used to solubilize or suspend the active agent(s). Such formulations also optionally include surfactants, which include anionic, nonionic, amphoteric and cationic surfactants. Such surfactants are known in the art as appropriate ingredients for mouthwashes.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Liquid formulations may contain additional components to improve the effectiveness of the product. For example, component(s) may be added to increase viscosity to provide improved retention on the surfaces of the oral cavity. Suitable viscosity increasing agents include carboxyalkyl, hydroxyalkyl, and hydroxyalkyl alkyl celluloses, xanthan gum, carageenan, alginates, pectins, guar gum, polyvinylpyrolidone, and gellan gums. High viscosity formulations may cause nausea in chemotherapy and radiation patients and are therefore not preferred. Gellan gums are sometimes preferred as viscosity modifying agents since aqueous solutions containing certain gellan gums may be prepared so that they will experience an increase in viscosity upon contact with electrolytes. Saliva contains electrolytes that may interact with such a gellan containing solution so as to increase its viscosity in situ.

In certain embodiments, compositions containing the second drug are film-forming or otherwise provide a coating effect on oral mucosa. In some cases they are formulated to contain a mucoadhesive polymer, a viscous polymer gel or a hydrogel, by adaptation of the materials and methods of WO 2004/032843. For instance, a formulation can contain a second drug and at least one cationic polymer or a neutral polymer that becomes cationic upon contact with an aqueous medium such as saliva, thus providing a mucoadhesive or gel forming material.

The cationic polymer can be any pharmaceutically acceptable natural or synthetic polymer which has the desired physical or chemical properties to enhance retention in the mouth. Polymers will typically be cationic polymers, mucoadhesive polymers or polymers which form a gel or hydrogel that physically adheres to the mucosa. In certain embodiments, the cationic polymer is a natural polymer such as gelatin or chitosan. Most synthetic polymers including a relatively high number of carboxylic groups will be mucoadhesive. In certain embodiments, compositions of the present invention comprise one or more natural polymers. Exemplary natural polymers include zein, modified zein, casein, gelatin, gluten, chitosan, collagen, polysaccharides such as cellulose, dextrans, polyhyaluronic acid, and alginic acid. In other embodiments, compositions of the present invention comprise one or more synthetic polymers. Exemplary synthetic polymers include poly(vinyl) alcohols, polyacrylamides, polyalkylene glycols, polyalkylene oxides, polyvinyl esters, PVP, alkyl cellulose (ethyl cellulose, methyl cellulose, etc.), hydroxyalkyl cellulose (e.g. hydroxypropyl cellulose, hydroxypropyl methyl cellulose, hydroxybutyl methyl celulose, etc.), and the like. Other suitable gel or hydrogel forming polymers are well known to one of ordinary skill in the art. Other suitable polymers are biodegradable. Suitable materials and methods are as disclosed in WO 2004/032843.

In some embodiments, the second drug is present in a composition in the form of a liquid bandage (see, e.g., U.S. Pat. Nos. 5,081,158; 5,166,233; 5,236,713 and 5,885,611). Such compositions can contain, in addition to a second drug of this invention, one or more of propylene glycol, polyvinylpyrrolidone (also known as povidone or PVP), or hyaluronic acid or a salt thereof, and optionally a flavoring agent and/or a local anaesthetic agent. In other embodiments, compositions of the present invention optionally further comprise glycyrrhetinic acid.

In certain embodiments, hyaluronic acid, or salt thereof, is present in weight percentages ranging from about 0.01 to about 5%. In other embodiments, hyaluronic acid, or salt thereof, is present in about 0.1%. According to another embodiment, glycyrrhetinic acid is present in an amount ranging from about 0.01 to 3% by weight. According to yet another embodiment, PVP is present in an amount ranging from about 1% to about 20% by weight.

“Liquid bandage” formulations based on a cellulose gelling agent are also of interest as a formulation for the second drug. For instance, such a liquid bandage formulation may contain ethyl cellulose from about 8 to about 12% (or greater) by weight in an ethyl alcohol vehicle (ethanol content from about 50 to about 60% by weight). Other vehicle materials can include purified water (about 5 to about 10% by weight) and propylene glycol (about 2 to about 5% by weight). Benzocaine or lidocaine (or the hydrochloride of either) can be included in amounts of about 10 to about 20% by weight as an optional anesthetic compound. Tannic acid may optionally be included as an astringent compound at a concentration of about 1 to about 5% by weight. Salicylic acid may be included an optional keratolytic agent at a concentration of about 1 to about 5% by weight.

The various formulations may also contain pharmaceutically inactive ingredients as for example, a sweetener such as sodium saccharin (0.1-1% by weight) and a flavorant (0.1-1% by weight) such as mint or menthol flavors. Flavorings used in the mouthrinse art such as peppermint, citrus flavorings, berry flavorings, vanilla, cinnamon, and sweeteners, either natural or artificial, may be used. Flavorings that are known to increase salivary electrolyte concentrations may be added to increase the magnitude of the viscosity change. The increased viscosity will promote retention of the solutions in the oral cavity and provide greater effectiveness due to increased contact time with the affected tissues.

In order to improve patient acceptability, it is desirable to add an appropriate coloring and/or flavoring material. Any pharmaceutically acceptable coloring or flavoring material may be used. Additionally, any of the mouthwash, mouthrinse, liquid bandage and other liquid compositions of this invention may be chilled to a temperature below body temperature, in some cases below room temperature, e.g. between 30° C. and 40° C., before administration to the patient.

Compositions of the present invention optionally further comprise one or more other active ingredients such as antibacterials, disinfectants, antifungals, analgesics, emollients, local anesthetics, and the like.

Additional antimicrobial preservatives may be component of the formulation in cases where it is necessary to inhibit microbial growth. Suitable preservatives include, but are not limited to the alkyl parabens, benzoic acid, and benzyl alcohol. The quantity of preservative may be determined by conducting standard antimicrobial preservative effectiveness tests such as that described in the United States Pharmacopoeia.

Suitable solid dosage forms include powders or tablets that are designed for constitution as solutions by dissolution or suspension in a liquid vehicle and include troches, pastilles, lollipop, or lozenges that dissolve slowly in the mouth. For convenience of use, solids designed to be dissolved to prepare a liquid dosage form prior to administration are rapidly dissolving. Technologies to produce rapidly dissolving solids are well known in the art. These include spray-drying, freeze-drying, particle size reduction and optimizing the pH of the dissolution medium.

In other embodiments, the dosage form is a concentrated gel that is optionally diluted, e.g., with water, prior to administration.

Other medicinal agents may be included in the regimen, e.g., in the composition containing the second drug, for purposes of alleviating other undesirable conditions in the mouth. Such agents may include, for example, local anesthetics, anti-infective agents, and emollients. Examples of local anesthetics are lidocaine and a eutectic mixture of lidocaine and prilocalne. Lidocaine may be administered in solution at a concentration of 2%, at a dose of 15 ml, at intervals of not less than three hours. The eutectic mixture is equimolar, administered at a total concentration of up to 5%. Either could be incorporated in an aerosol at similar doses.

The various compositions may include additional ingredients, such as analgesics for pain relief, antibiotics to lower the risk of or to treat infection, and other agents which might help treat mucositis or promote wound healing. Liquid compositions of the invention may further include a thickening or adhesive agent such as a mucosal-adhesive water-soluble polymer or biocompatible reverse-thermal gelation polymer to help prolong the contact of mucosa with drug, alleviate pain and/or avoid infection. In some other embodiments, the composition may include an agent which promotes cell penetration by the drug.

5. Doses and Routes of Administration

The second drug according to the method of the present invention may be administered using parenteral or topical administration in any amount effective for preventing, treating or lessening the severity of mouth sores Induced by the administration of an mTOR inhibitor. The exact amount required will vary from subject to subject, depending on the age, and general condition of the subject, the severity of the infection, the particular agent, its formulation, and the like.

For parenteral administration of the second drug of this invention, the dose level and dose frequency of current interest include those approved for monotherapy or other combination therapies involving administration of the respective second drug, as well as doses effective to reduce the incidence or severity of mouth sores which can accompany the administration of an mTOR inhibitor. For example, and effective amount of the second drug is from 0.01 mg/kg to 100 mg/kg, from 0.01 mg/kg to 50 mg/kg, from 0.1 mg/kg to 30 mg/kg, from 0.1 mg/Kg to 20 mg/Kg and from 0.5 mg/Kg to 10 mg/Kg of patient body weight, depending in part upon the potency of the second drug chosen. By way of example, in the case of Remicade, the dose approved for systematic administration is 3 mg/Kg to 5 mg/Kg of patient body weight. In the case where the second drug is Raptiva, the dose approved for systemic administration is 0.7 mmg/Kg of patient body weight.

The dose frequency of the administration also depends on the nature of the second drug. The dose frequency of current interest for parenteral administration of the second drug is the dose frequency approved for monotherapy or other combination therapies involving administration of the respective second drug. The second drug may be administered once or multiple times daily, weekly or at some other multiple day/week interval, or on an intermittent schedule. For example, the second drug may be administered twice weekly, every week, every other week or every 4 weeks. The second drug may also be administered twice a week, every week or every other week for a period of weeks (e.g. 2-4 weeks) followed by a maintenance dose on a less frequent schedule (e.g. every 4 weeks, or every 8 weeks).

For topical administration of the second drug of the invention, the dose level of current interest is 0.3 to 5 times the dose levels approved or studied for the respective agent when given systemically. For example, the dose level for topical administration is typically from 0.001-200 mg/kg, from 0.01-150 mg/Kg, from 0.1-100 mg/Kg, from 0.3-100 mmg/Kg and from 0.5-50 mg/kg of patient body weight. The effective amount will depend upon the potency of the drug chosen, the nature of the vehicle and excipients and the frequency of administration.

In the case of topical administration of the second drug, e.g. in a mouth wash, mouth rinse, mouth spray, among others, the extended topical exposure of the drug is at least half a minute, preferably 1 to 5 minutes or longer. Further extended durations of topical exposure may be beneficial, e.g. for 5-10 minutes or for tens of minutes, especially in cases where the formulation doesn't contain an agent for prolonging the topical exposure e.g. a gelling or adhesive agent.

The mTOR inhibitor according to the method of the present invention may be administered using any amount and any route of administration effective for treating, preventing and/or managing various types of cancers. The exact amount required will also vary from subject to subject, depending on age, and general condition of the subject, the severity of the cancer, the particular mTOR inhibitor, its mode of administration, and the like.

The specific effective dose level of mTOR inhibitor and of the second drug of the invention for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

It will be understood, however, that the total daily or weekly usage of the compounds and compositions of the present invention will be decided by the attending physician within the scope of sound medical judgment.

6. Additional Drug Combinations

It will also be appreciated that rapamycin analog/second drug combinations of the present invention can be employed in further combination with additional therapies (i.e., a treatment according to the present invention can be administered concurrently with, prior to, or subsequently to one or more desired therapeutics or medical procedures). The particular combination of therapies (therapeutics or procedures) to employ in such a further recombination regimen will take into account compatibility of the desired therapeutics and/or procedures and the desired therapeutic effect to be achieved.

For example, methods and compositions of the present invention can be employed together with other procedures including surgery, radiotherapy (e.g., g-radiation, neuron beam radiotherapy, electron beam radiotherapy, proton therapy, brachytherapy, an systemic radioactive isotopes), endocrine therapy, hyperthermia and cryotherapy.

Alternatively or additionally, methods and compositions of the present invention can be employed together with other agents to attenuate any adverse effects (e.g., statins, pain medication, antiemetics, G-CSF, GM-CSF, etc.), and/or with other approved chemotherapeutic drugs. Such other drugs include but not limited to one or more of the following: an anti-cancer alkylating or intercalating agent (e.g., mechlorethamine, chlorambucil, Cyclophosphamide, Melphalan, and Ifosfamide); antimetabolite (e.g., Methotrexate); purine antagonist or pyrimidine antagonist (e.g., 6-Mercaptopurine, 5-Fluorouracil, Cytarablle, capecitabine and Gemcitabine); spindle poison (e.g., Vinblastine, Vincristine, Vinorelbine and Paclitaxel); podophyllotoxin (e.g., Etoposide, Irinotecan, Topotecan); antibiotic (e.g., Doxorubicin, Bleomycin and Mitomycin); nitrosourea (e.g., Carmustine, Lomustine); inorganic ion (e.g., Cisplatin, Carboplatin, Oxaliplatin or oxiplatin); enzyme (e.g., Asparaginase); hormone (e.g., Tamoxifen, Leuprolide, Flutamide and Megestrol); proteasome inhibitor (such as Velcade, another proteasome inhibitor (see e.g., WO 02/096933) or another NF-kB inhibitor, including, e.g., an IkK inhibitor); other kinase inhibitors (e.g., an inhibitor of Src, BRC/Abl, kdr, flt3, aurora-2, glycogen synthase kinase 3 (“GSK-3”), EGF-R kinase (e.g., Iressa, Tarceva, etc.), VEGF-R kinase, PDGF-R kinase, etc); an antibody, soluble receptor or other receptor antagonist against a receptor or hormone implicated in a cancer (Including receptors such as EGFR, ErbB2, VEGFR, PDGFR, and IGF-R; and agents such as Herceptin (or other anti-Her2 antibody), Avastin, Erbitux, etc.); etc. For a more comprehensive discussion of updated cancer therapies see, http://www.nci.nih.gov/, a list of the FDA approved oncology drugs at http://www.fda.gov/cder/cancer/druglistframe.htm, and The Merck Manual, Seventeenth Ed. 1999, the entire contents of which are hereby incorporated by reference. Examples of other therapeutic agents include among others, Zyloprim, alemtuzumab, altretamine, amifostine, nastrozole, antibodies against prostate-specific membrane antigen (such as MLN-591, MLN591RL and MLN2704), arsenic trioxide, bexarotene, bleomycin, busulfan, capecitabine, Gliadel Wafer, celecoxib, chlorambucil, cisplatin-epinephrine gel, cladribine, cytarabine liposomal, daunorubicin liposomal, daunorubicin, daunomycin, dexrazoxane, docetaxel, doxorubicin, Elliott's B Solution, epirubicin, estramustine, etoposide phosphate, etoposide, exemestane, fludarabine, 5-FU, fulvestrant, gemcitabine, gemtuzumab-ozogamicin, goserelin acetate, hydroxyurea, idarubicin, idarubicin, Idamycin, ifosfamide, imatinib mesylate, irinotecan (or other topoisomerase inhibitor, including antibodies such as MLN576 (XR11576)), letrozole, leucovorin, leucovorin levamisole, liposomal daunorubicin, melphalan, L-PAM, mesna, methotrexate, methoxsalen, mitomycin C, mitoxantrone, MLN518 or MLN608 (or other inhibitors of the fit-3 receptor tyrosine kinase, PDFG-R or c-kit), itoxantrone, paclitaxel, Pegademase, pentostatin, porfimer sodium, Rituximab (RITUXAN®), talc, tamoxifen, temozolamide, teniposide, VM-26, topotecan, toremifene, 2C4 (or other antibody which interferes with HER2-mediated signaling), tretinoin, ATRA, valrubicin, vinorelbine, or pamidronate, zoledronate or another bisphosphonate.

The combination therapy of this invention can also be employed together with one or more further combinations of cytotoxic agents as part of a treatment regimen, wherein the further combination of cytotoxic agents is selected from: CHOPP (cyclophosphamide, doxorubicin, vincristine, prednisone, and procarbazine); CHOP (cyclophosphamide, doxorubicin, vincristine, and prednisone); COP (cyclophosphamide, vincristine, and prednisone); CAP-BOP (cyclophosphamide, doxorubicin, procarbazine, bleomycin, vincristine, and prednisone); m-BACOD (methotrexate, bleomycin, doxorubicin, cyclophosphamide, vincristine, dexamethasone, and leucovorin); ProMACE-MOPP (prednisone, methotrexate, doxorubicin, cyclophosphamide, etoposide, leucovorin, mechloethamine, vincristine, prednisone, and procarbazine); ProMACE-CytaBOM (prednisone, methotrexate, doxorubicin, cyclophosphamide, etoposide, leucovorin, cytarabine, bleomycin, and vincristine); MACOP-B (methotrexate, doxorubicin, cyclophosphamide, vincristine, prednisone, bleomycin, and leucovorin); MOPP (mechloethamine, vincristine, prednisone, and procarbazine); ABVD (adriamycin/doxorubicin, bleomycin, vinblastine, and dacarbazine); MOPP (mechloethamine, vincristine, prednisone and procarbazine) alternating with ABV (adriamycin/doxorubicin, bleomycin, and vinblastine); MOPP (mechloethamine, vincristine, prednisone, and procarbazine) alternating with ABVD (adriamycin/doxorubicin, bleomycin, vinblastine, and dacarbazine); ChlVPP (chlorambucil, vinblastine, procarbazine, and prednisone); IMVP-16 (ifosfamide, methotrexate, and etoposide); MIME (methyl-gag, ifosfamide, methotrexate, and etoposide); DHAP (dexamethasone, high-dose cytaribine, and cisplatin); ESHAP (etoposide, methylpredisolone, high-dose cytarabine, and cisplatin); CEPP(B) (cyclophosphamide, etoposide, procarbazine, prednisone, and bleomycin); CAMP (lomustine, mitoxantrone, cytarabine, and prednisone); CVP-1 (cyclophosphamide, vincristine, and prednisone), ESHOP (etoposide, methylpredisolone, high-dose cytarabine, vincristine and cisplatin); EPOCH (etoposide, vincristine, and doxorubicin for 96 hours with bolus doses of cyclophosphamide and oral prednisone), ICE (ifosfamide, cyclophosphamide, and etoposide), CEPP(B) (cyclophosphamide, etoposide, procarbazine, prednisone, and bleomycin), CHOP-B (cyclophosphamide, doxorubicin, vincristine, prednisone, and bleomycin), CEPP-B (cyclophosphamide, etoposide, procarbazine, and bleomycin), and P/DOCE (epirubicin or doxorubicin, vincristine, cyclophosphamide, and prednisone).

The following examples contain additional information, exemplification and guidance which can be adapted to the practice of this invention in its various embodiments and the equivalents thereof. The examples are intended to help illustrate the invention, and are not intended to, nor should they be construed to, limit its scope in any way. Indeed, various modification of the invention, and many further embodiments thereof, in addition to those shown and described herein, will be apparent to those skilled in the art upon review of this document, including the examples which follow and the references to the scientific and patent literature cited herein. Such modifications and variations, including design choices in selecting, preparing, formulating and administering the mTOR inhibitor or the second drug of this invention, etc. are intended to be encompassed by the scope of the invention and of the appended claims.

### EXAMPLES

### Example 1

### Formulation of the Second Drug for Local/Topical Delivery

The formulations may be prepared as a liquid, semi-solid, or solid containing an amount of the second drug of the invention that is effective to treat or prevent mouth sores. Generally, these compositions contain about 0.1-800 mg/mL, 0.1-500 mg/mL, 0.5-500 mg/mL, 1-500 mg/mL, 10-400 mg/mL, 25-300 mg/mL, 50-250 mg/mL or 50-100 mg/mL of the second drug. The effective amount of the second drug will depend upon the potency of the compound chosen, the nature of the vehicle and excipients, and the frequency of administration. By way of example, in the case of Enbrel, the composition generally contains a dose in a range of 15-250 mg/mL, often 40-80 mg/mL of the compound.

A. Buffered Solutions

Solutions of the second drug can be prepared by mixing the compound to a concentration of 0.1-800 mg/mL, 0.1-500 mg/mL, 0.5-500 mg/mL, 1-500 mg/mL, 10-400 mg/mL, 25-300 mg/mL, 50-250 mg/mL or 50-100 mg/mL with a solution containing methyl and propyl parabens as antimicrobial preservatives at concentrations of 0.18% and 0.02%, respectively and tromethamine buffer.

B. Gellan Gum Buffered Formulations.

Formulations of the second drug can be prepared by adding the compound to a solution containing gellan gum at a concentration of 0.5 mg/mL. The concentration of the second drug can vary between 0.1-800 mg/mL, 0.1-500 mg/mL, 0.5-500 mg/mL, 1-500 mg/ml, 10-400 mg/mL, 25-300 mg/mL, 50-250 mg/mL or 50-100 mg/mL depending on the specific chosen compound. The solution also contains methyl and propyl parabens as antimicrobial preservatives at concentrations of 0.18% and 0.02%, respectively and tromethamine buffer.

C. Oral Rinses.

A mixture of a selected second drug and a buffer to promote rapid dissolution is subjected to granulation. The resultant material is dissolved in water to form an oral rinse solution containing 0.1-800 mg/mL, 0.1-500 mg/mL, 0.5-500 mg/mL, 1-500 mg/mL, 10-400 mg/mL, 25-300 mg/mL, 50-250 mg/mL or 50-100 mg/mL of the compound per mL of formulation.

D. Effervescent Tablets.

Effervescent tablets can be prepared by mixing a selected second drug with sodium bicarbonate. The tablet is then dissolved in water to form an oral rinse solution containing 0.1-800 mg/mL, 0.1-500 mg/mL, 0.5-500 mg/mL, 1-500 mg/mL, 10-400 mg/mL, 25-300 mg/mL, 50-250 mg/mL or 50-100 mg/mL.

E. Rapidly Disintegrating Formulations

Rapidly disintegrating formulations of the second drug may also be prepared as described in WO 2004/000223, substituting the second drug in accordance with this invention for the tetracycline compound disclosed there.

### Example 2

### Formulation 02 of the second drug

In another approach, the second drug is formulated with a biocompatible reverse-thermal gelation polymer using the materials and methods of WO 02/41837.

### Example 3

### Formulation 03 of the second drug

In another approach, the second drug is administered in a concentrated oral gel formulation. In this case, the second drug in a concentration of 0.1-800 mg/mL, 0.1-500 mg/mL, 0.5-500 mg/mL, 1-500 mg/mL, 10-400 mg/mL, 25-300 mg/mL, 50-250 mg/mL or 50-100 mg/mL is combined with the contents of one packet of Gelclairn™ (OSI Pharmaceuticals) and one tablespoon of water and stirred well. The mixture is used to rinse the mouth for at least 1 minute or as long as possible to coat the tongue, palate, throat, inside of cheeks and all oral tissue well. The material is gargled and then spit out, and administration is repeated 3 times per day or as needed, all in accordance with the normal directions for use of the Gelclair product.

### Example 4

### Formulation 04 of the second drug

In another approach, the second drug of the invention can be stirred into a glass of water to which a dose of AlkaSelzer is added and the mixture is swishes and spit out or swishes and ingested, in accordance with the normal directions for the use of alka selzer.

### Example 5

### Formulation of a rapamycin analog (AP23573)

The following procedure was used to prepare a tablet containing 10 mg of AP23573 and containing the following components. The tablets are coated with two different coatings—a film-coated tablet for immediate release and an enteric-coated tablet for delayed release. The composition of the core tablet is shown in the following table. Core tablets are film-coated and may be used as such, or may be enteric-coated.

| Component | Weight Percent |
|---|---|
| AP23573 | 8.00% |
| Butylated Hydroxytoluene | 0.08% |
| Hydroxy Propyl Cellulose | 8% |
| Lactose Monohydrate | 50.57% |
| Microcrystalline Cellulose | 30.85% |
| Croscarmellose Sodium | 2.00% |
| Magnesium Stereate | 0.50% |
| Dehydrated Alcohol (Ethanol)* | — |

*may be used in processing but may not appear in final product

Hydroxypropyl Cellulose, Lactose Monohydrate, Microcrystalline Cellulose, and half of the Croscarmellose Sodium, were mixed in a high shear granulator. The AP23573 and Butylated Hydroxytoluene (BHT) were dissolved in Dehydrated Alcohol, USP, mixing not less than 45 minutes. The solution of AP23573 and BHT was added to the granulator and mixed to a wet mass for approximately 3 minutes.

The granulation was dried in a fluid bed dryer at 45-55° C. for 60-90 minutes, after which the dried granulation was passed through a mill fitted with a 0.045-inch screen opening to remove oversized granulation. The milled granulation was then blended with Magnesium Stearate, NF and the remaining half of the Croscarmellose Sodium, NF.

The granulation was pressed into tablets using a tablet press set up with 6 mm round concave tooling. The press was adjusted as required for a target tablet weight of 125.0 mg, hardness of 5.5 kp, friability no more than 1%, and disintegration time less than 10 minutes.

### Film Coating

A film coating may be prepared according to following procedure using the following components. The tablets are added to a coating pan and are coated with a solution of Copovidone in Dehydrated Alcohol, USP (20:80 w/w), maintaining a product temperature of 20-35° C., until a weight gain of 5% is achieved. The pan is then cooled and the film-coated tablets allowed to dry. Film-coated tablets may be packaged as such, or may be enteric coated.

### Enteric Coating

An enteric coating may be prepared according to following procedure using the following components.

| Film Coating | Percent of Suspension |
|---|---|
| Methacrytic Acid Copolymer | 11.03% |
| Triethyl Citrate | 2.16% |
| Talc | 2.81% |
| Dehydrated Alcohol (Ethanol)* | 84.00% |

*Use in processing but not necessarily retained in final product

For enteric coating, the tablets are placed in a coating pan and coated with a suspension of Methacrylic Acid Copolymer, NF, Triethyl Citrate, NF, and Talc in Dehydrated Alcohol, USP, maintaining a product temperature of 20-35° C., until a weight gain of 8% is achieved. The pan is then cooled, and the enteric-coated tablets allowed to dry.

### Example 6

### Methods of Treatment

1. Intravenous administration of Infliximab in combination with AP23573 in the treatment of refractory or advanced cancers
Infliximab is infused intravenously into an adult patient in a 5 mg/kg dose, two weeks prior the first administration of AP23573. The initial dose is then followed by additional 5 mg/Kg doses two and 6 weeks after the first dose. A maintenance dose of 5 mg/Kg is infused every eight weeks thereafter.
Two weeks after the first infliximab infusion, the patient begins a repeating cycle of daily dosing of AP23573 on one of the following schedules:
(a) 30 mg of AP23573 orally every day for six consecutive days, followed by 1, 8, 15 or 22 days without additional AP23573, then starting the next cycle of treatment with AP23573.
(b) 30 mg of AP23573 orally every day for five consecutive days, followed by 2, 9, 16 or 23 days without additional AP23573, then starting the next cycle of treatment with AP23573.
(c) 10-40 mg of AP23573 orally every day for 28 days, then starting the next cycle without interruption of treatment.
(d) 10-40 mg of AP25373 orally every day for 21 days, followed by 7 days without additional AP23573, then starting the next cycle of treatment with AP23573.
(e) 40-80 mg of AP23573 administered orally in any of the schedule a through d.
2. Intravenous administration of Infliximab in combination with intravenous AP23573 in the treatment of refractory or advanced cancers
Infliximab is infused intravenously into an adult patient in a 5 mg/kg dose, two weeks prior the first administration of AP23573. The initial dose is then followed by additional 5 mg/Kg doses two and 6 weeks after the first dose. A maintenance dose of 5 mg/Kg is infused every eight weeks thereafter.
Two weeks after the first infliximab infusion, the patient begins a repeating cycle of intravenous dosing of AP23573 on one of the following schedules:
(a) 10-40 mg of AP23573 injected every day for 5 consecutive days, followed by 2 or 9 days without additional AP23573, then starting the next cycle of treatment with AP23573.
(b) 40-80 mg of AP25373 injected every day for 5 consecutive days, followed by 2 or 9 days without additional AP23573, then starting the next cycle of treatment with AP23573.
3. Intravenous administration of Infliximab in combination with oral temsirolimus in the treatment of refractory or advanced cancers
Infliximab is infused intravenously into an adult patient in a 5 mg/kg dose, two weeks prior the first administration of temsirolimus. The initial dose is then followed by additional 5 mg/Kg doses two and 6 weeks after the first dose. A maintenance dose of 5 mg/Kg is infused every eight weeks thereafter.
Two weeks after the first infliximab infusion, the patient begins a repeating cycle of daily dosing of temsirolimus on one of the following schedules:
(a) 20 mg of temsirolimus orally every day for six consecutive days, followed by 1, 8, 15 or 22 days without additional temsirolimus, then starting the next cycle of treatment with temsirolimus.
(b) 30-80 mg of temsirolimus orally every day for six consecutive days, followed by 1, 8, 15 or 22 days without additional temsirolimus, then starting the next cycle of treatment with Temsirolimus.
(c) any of the previous doses in schedule a and b, where Temsirolimus is administered orally every day for five consecutive days, followed by 2, 9, 16 or 23 days without additional Temsirolimus, then starting the next cycle of treatment with Temsirolimus.
(d) 30-75 mg of Temsirolimus orally every day for five consecutive days, every 2 weeks.
(e) 30-75 mg of Temsirolimus orally every day for 7 consecutive days, followed by 8 days without additional Temsirolimus, then starting the next cycle of treatment with Temsirolimus.
4. Intravenous administration of Infliximab in combination with intravenous temsirolimus in the treatment of refractory or advanced cancers
Infliximab is infused intravenously into an adult patient in a 5 mg/kg dose, two weeks prior the first administration of temsirolimus. The initial dose is then followed by additional 5 mg/Kg doses two and 6 weeks after the first dose. A maintenance dose of 5 mg/Kg is infused every eight weeks thereafter.

Two weeks after the first infliximab infusion, the patient begins a repeating cycle of intravenous dosing of temsirolimus on one of the following schedules:

(a) 75 mg of temsirolimus in 30 minutes intravenous infusion on days 1, 8, 15 and 22 (i.e. day 1 being 2 weeks after the first infusion of Infliximab). In the absence of disease progression and unacceptable toxicity, the cycle repeats every 28 days.
(b) 75-250 mg of temsirolimus in 30 minutes intravenous infusion on days 1, 8, 15 and 22 (i.e. day 1 being 2 weeks after the first infusion of Infliximab). In the absence of disease progression and unacceptable toxicity, the cycle repeats every 28 days.
(c) 250 mg and higher doses of temsirolimus in 30 minutes intravenous infusion on days 1, 8, 15 and 22 (i.e. day 1 being 2 weeks after the first infusion of Infliximab). In the absence of disease progression and unacceptable toxicity, the cycle repeats every 28 days.
5. Intravenous administration of Infliximab in combination with Everolimus in the treatment of refractory or advanced cancers
Infliximab is infused intravenously into an adult patient in a 5 mg/kg dose, two weeks prior the first administration of everolimus. The initial dose is then followed by additional 5 mg/Kg doses two and 8 weeks after the first dose. A maintenance dose of 5 mg/Kg is infused every eight weeks thereafter.

Two weeks after the first Infliximab infusion, the patient begins a repeating cycle of daily dosing of Everolimus on one of the following schedules:

(a) 1.5 to 5 mg of Everolimus orally every day every day for five or six consecutive days, followed by 1, 8, 15 or 22 days without additional Everolimus, then starting the next cycle of treatment with Everolimus.
(b) 10-20 mg of Everolimus orally every day for six consecutive days, followed by 1, 8, 15 or 22 days without additional Everolimus, then starting the next cycle of treatment with Everolimus.
(c) 20 mg and higher doses of Everolimus orally every day for six consecutive days, followed by 1, 8, 15 or 22 days without additional Everolimus, then starting the next cycle of treatment with Everolimus.
(d) any of the previous doses in schedule a through c, where Everolimus is administered orally every day for five consecutive days, followed by 2, 9, 16 or 23 days without additional Everolimus, then starting the next cycle of treatment with Everolimus.
(e) any of the previous doses in schedule a through c, where Everolimus is administered orally once daily for 28 days. In the absence of disease progression and unacceptable toxicity, the treatment continues for an additional 28 days and so on.
6. Subcutaneous administration of Etanercept in combination with AP23573 in the treatment of refractory or advanced cancers
Etanercept is injected subcutaneously into an adult patient in a 50 mg dose, two weeks prior the first administration of AP23573. The initial dose is then followed by additional 50 mg doses twice a week for 3 months. A maintenance dose of 50 mg is injected every week thereafter.
Two weeks after the first Etanercept injection, the patient begins a repeating cycle of daily dosing of AP23573 on one of the following schedules:
(a) 30 mg of AP23573 orally every day for six consecutive days, followed by 1, 8, 15 or 22 days without additional AP23573, then starting the next cycle of treatment with AP23573.
(b) 30 mg of AP23573 orally every day for five consecutive days, followed by 2, 9, 16 or 23 days without additional AP23573, then starting the next cycle of treatment with AP23573.
(c) 40-80 mg of AP23573 orally every day for five consecutive days, followed by 2, 9, 16 or 23 days without additional AP23573, then starting the next cycle of treatment with AP23573.
(d) 40-80 mg of AP23573 orally every day for six consecutive days, followed by 1, 8, 15 or 22 days without additional AP23573, then starting the next cycle of treatment with AP23573.
7. Subcutaneous administration of Etanercept in combination with intravenous AP23573 in the treatment of refractory or advanced cancers
Etanercept is injected subcutaneously into an adult patient in a 50 mg dose, two weeks prior the first administration of AP23573. The initial dose is then followed by additional 50 mg doses twice a week for 3 months. A maintenance dose of 50 mg is injected every week thereafter.
Two weeks after the first Etanercept injection, the patient begins a repeating cycle of daily dosing of AP23573 on one of the following schedules:
(a) 10-40 mg of AP23573 injected every day for 5 consecutive days, followed by 2 or 9 days without additional AP23573, then starting the next cycle of treatment with AP23573.
(b) 40-80 mg of AP25373 injected every day for 5 consecutive days, followed by 2 or 9 days without additional AP23573, then starting the next cycle of treatment with AP23573.
8. Subcutaneous administration of Etanercept in combination with oral Temsirolimus in the treatment of refractory or advanced cancers
Etanercept is injected subcutaneously into an adult patient in a 50 mg dose, two weeks prior the first administration of AP23573. The initial dose is then followed by additional 50 mg doses twice a week for 3 months. A maintenance dose of 50 mg is injected every week thereafter.
Two weeks after the first Etanercept injection, the patient begins a repeating cycle of daily dosing of Temsirolimus on one of the following schedules:
(a) 20 mg of Temsirolimus orally every day for six consecutive days, followed by 1, 8, 15 or 22 days without additional Temsirolimus, then starting the next cycle of treatment with Temsirolimus.
(b) 30-80 mg of Temsirolimus orally every day for six consecutive days, followed by 1, 8, 15 or 22 days without additional Temsirolimus, then starting the next cycle of treatment with Temsirolimus.
(c) any of the previous doses in schedule a and b, where Temsirolimus is administered orally every day for five consecutive days, followed by 2, 9, 16 or 23 days without additional Temsirolimus, then starting the next cycle of treatment with Temsirolimus.
(d) 30-75 mg of Temsirolimus orally every day for five consecutive days, every 2 weeks.
(e) 30-75 mg of Temsirolimus orally every day for 7 consecutive days, followed by 8 days without additional Temsirolimus, then starting the next cycle of treatment with Temsirolimus.
9. Subcutaneous administration of Etanercept in combination with intravenous Temsirolimus in the treatment of refractory or advanced cancers
Etanercept is injected subcutaneously into an adult patient in a 50 mg dose, two weeks prior the first administration of AP23573. The initial dose is then followed by additional 50 mg doses twice a week for 3 months. A maintenance dose of 50 mg is injected every week thereafter.
Two weeks after the first Etanercept injection, the patient begins a repeating cycle of daily dosing of Temsirolimus on one of the following schedules:
(a) 75 mg of Temsirolimus in 30 minutes intravenous infusion on days 1, 8, 15 and 22 (i.e. day 1 being 2 weeks after the first infusion of Etanercept). In the absence of disease progression and unacceptable toxicity, the cycle repeats every 28 days.
(b) 75-250 mg of Temsirolimus in 30 minutes intravenous infusion on days 1, 8, 15 and 22 (i.e. day 1 being 2 weeks after the first infusion of Etanercept). In the absence of disease progression and unacceptable toxicity, the cycle repeats every 28 days.
(c) 250 mg and higher doses of Temsirolimus in 30 minutes intravenous infusion on days 1, 8, 15 and 22 (i.e. day 1 being 2 weeks after the first infusion of Etanercept). In the absence of disease progression and unacceptable toxicity, the cycle repeats every 28 days.
10. Subcutaneous administration of Etanercept in combination with Everolimus in the treatment of refractory or advanced cancers
Etanercept is injected subcutaneously into an adult patient in a 50 mg dose, two weeks prior the first administration of AP23573. The initial dose is then followed by additional 50 mg doses twice a week for 3 months. A maintenance dose of 50 mg is injected every week thereafter.
Two weeks after the first Etanercept injection, the patient begins a repeating cycle of daily dosing of everolimus on one of the following schedules:
(a) 1.5 to 5 mg of Everolimus orally every day every day for six consecutive days, followed by 1, 8, 15 or 22 days without additional Everolimus, then starting the next cycle of treatment with Everolimus.
(b) 10-20 mg of Everolimus orally every day for six consecutive days, followed by 1, 8, 15 or 22 days without additional Everolimus, then starting the next cycle of treatment with everolimus.
(c) 20 mg and higher doses of Everolimus orally every day for six consecutive days, followed by 1, 8, 15 or 22 days without additional Everolimus, then starting the next cycle of treatment with Everolimus.
(d) any of the previous doses in schedule a through c, where Everolimus is administered orally every day for five consecutive days, followed by 2, 9, 18 or 23 days without additional Everolimus, then starting the next cycle of treatment with Everolimus.
(e) any of the previous doses in schedule a through c, where Everolimus is administered orally once daily for 28 days. In the absence of disease progression and unacceptable toxicity, the treatment continues for an additional 28 days and so on.
11. Subcutaneous administration of Humira in combination with AP23573 in the treatment of refractory or advanced cancers
Humira is injected subcutaneously into an adult patient in a 40 mg dose every other week, beginning two weeks prior the first administration of AP23573.
Two weeks after the first Humira injection, the patient begins a repeating cycle of daily dosing of AP23573 on one of the following schedules:
(a) 30 mg of AP23573 orally every day for six consecutive days, followed by 1, 8, 15 or 22 days without additional AP23573, then starting the next cycle of treatment with AP23573.
(b) 30 mg of AP23573 orally every day for five consecutive days, followed by 2, 9, 16 or 23 days without additional AP23573, then starting the next cycle of treatment with AP23573.
(c) 40-80 mg of AP23573 orally every day for five consecutive days, followed by 2, 9, 16 or 23 days without additional AP23573, then starting the next cycle of treatment with AP23573.
(d) 40-80 mg of AP23573 orally every day for six consecutive days, followed by 1, 8, 15 or 22 days without additional AP23573, then starting the next cycle of treatment with AP23573.
12. Subcutaneous administration of Humira In combination with intravenous AP23573 in the treatment of refractory or advanced cancers
Humira is injected subcutaneously into an adult patient in a 40 mg dose every other week, beginning two weeks prior the first administration of AP23573.
Two weeks after the first Humira injection, the patient begins a repeating cycle of daily dosing of AP23573 on one of the following schedules:
(a) 10-40 mg of AP23573 injected every day for 5 consecutive days, followed by 2 or 9 days without additional AP23573, then starting the next cycle of treatment with AP23573.
(b) 40-80 mg of AP25373 injected every day for 5 consecutive days, followed by 2 or 9 days without additional AP23573, then starting the next cycle of treatment with AP23573.
13. Subcutaneous administration of Humira in combination with oral Temsirolimus in the treatment of refractory or advanced cancers
Humira is injected subcutaneously into an adult patient in a 40 mg dose every other week, beginning two weeks prior the first administration of Temsirolimus.
Two weeks after the first Humira injection, the patient begins a repeating cycle of daily dosing of Temsirolimus on one of the following schedules:
(a) 20 mg of Temsirolimus orally every day for six consecutive days, followed by 1, 8, 15 or 22 days without additional Temsirolimus, then starting the next cycle of treatment with Temsirolimus.
(b) 30-80 mg of Temsirolimus orally every day for six consecutive days, followed by 1, 8, 15 or 22 days without additional Temsirolimus, then starting the next cycle of treatment with Temsirolimus.
(c) any of the previous doses in schedule a and b, where Temsirolimus is administered orally every day for five consecutive days, followed by 2, 9, 16 or 23 days without additional Temsirolimus, then starting the next cycle of treatment with Temsirolimus.
(d) 30-75 mg of Temsirolimus orally every day for five consecutive days, every 2 weeks.
(e) 30-75 mg of Temsirolimus orally every day for 7 consecutive days, followed by 8 days without additional Temsirolimus, then starting the next cycle of treatment with Temsirolimus.
14. Subcutaneous administration of Humira in combination with intravenous Temsirolimus in the treatment of refractory or advanced cancers
Humira is injected subcutaneously into an adult patient in a 40 mg dose every other week, beginning two weeks prior the first administration of Temsirolimus.
Two weeks after the first Humira injection, the patient begins a repeating cycle of daily dosing of Temsirolimus on one of the following schedules:
(a) 75 mg of Temsirolimus in 30 minutes intravenous infusion on days 1, 8, 15 and 22 (i.e. day 1 being 2 weeks after the first infusion of Humira). In the absence of disease progression and unacceptable toxicity, the cycle repeats every 28 days.
(b) 75-250 mg of Temsirolimus in 30 minutes intravenous infusion on days 1, 8, 15 and 22 (i.e. day 1 being 2 weeks after the first infusion of Humira). In the absence of disease progression and unacceptable toxicity, the cycle repeats every 28 days.
(c) 250 mg and higher doses of Temsirolimus in 30 minutes intravenous infusion on days 1, 8, 15 and 22 (i.e. day 1 being 2 weeks after the first infusion of Humira). In the absence of disease progression and unacceptable toxicity, the cycle repeats every 28 days.
15. Subcutaneous administration of Humira in combination with Everolimus in the treatment of refractory or advanced cancers
Humira is injected subcutaneously into an adult patient in a 40 mg dose every other week, beginning two weeks prior the first administration of Temsirolimus.
Two weeks after the first Humira injection, the patient begins a repeating cycle of daily dosing of everolimus on one of the following schedules:
(a) 1.5 to 5 mg of Everolimus orally every day every day for six consecutive days, followed by 1, 8, 15 or 22 days without additional Everolimus, then starting the next cycle of treatment with Everolimus.
(b) 10-20 mg of Everolimus orally every day for six consecutive days, followed by 1, 8, 15 or 22 days without additional Everolimus, then starting the next cycle of treatment with everolimus.
(c) 20 mg and higher doses of Everolimus orally every day for six consecutive days, followed by 1, 8, 15 or 22 days without additional Everolimus, then starting the next cycle of treatment with Everolimus.
(d) any of the previous doses in schedule a through c, where Everolimus is administered orally every day for five consecutive days, followed by 2, 9, 16 or 23 days without additional Everolimus, then starting the next cycle of treatment with Everolimus.
(e) any of the previous doses in schedule a through c, where Everolimus is administered orally once daily for 28 days. In the absence of disease progression and unacceptable toxicity, the treatment continues for an additional 28 days and so on.
Note: in Examples 11-15, Some patients may derive benefit from increasing the dosing of Humira to 40 mg every week.
16. Subcutaneous administration of Efalizumab in combination with AP23573 in the treatment of refractory or advanced cancers
Efalizumab is injected subcutaneously into an adult patient at a dose of 0.7 mg/kilogram of body weight, followed by weekly doses of 1 mg per kilogram (up to a maximum single dose of 200 mg), beginning two weeks prior the first administration of AP23573.
Two weeks after the first Efalizumab injection, the patient begins a repeating cycle of daily dosing of AP23573 on one of the following schedules:
(a) 30 mg of AP23573 orally every day for six consecutive days, followed by 1, 8, 15 or 22 days without additional AP23573, then starting the next cycle of treatment with AP23573.
(b) 30 mg of AP23573 orally every day for five consecutive days, followed by 2, 9, 16 or 23 days without additional AP23573, then starting the next cycle of treatment with AP23573.
(c) 40-80 mg of AP23573 orally every day for five consecutive days, followed by 2, 9, 16 or 23 days without additional AP23573, then starting the next cycle of treatment with AP23573.
(d) 40-80 mg of AP23573 orally every day for six consecutive days, followed by 1, 8, 15 or 22 days without additional AP23573, then starting the next cycle of treatment with AP23573.
17. Subcutaneous administration of Efalizumab in combination with intravenous AP23573 in the treatment of refractory or advanced cancers
Efalizumab is injected subcutaneously into an adult patient at a dose of 0.7 mg/kilogram of body weight, followed by weekly doses of 1 mg per kilogram (up to a maximum single dose of 200 mg), beginning two weeks prior the first administration of AP23573.
Two weeks after the first Efalizumab injection, the patient begins a repeating cycle of daily dosing of AP23573 on one of the following schedules:
(a) 10-40 mg of AP23573 injected every day for 5 consecutive days, followed by 2 or 9 days without additional AP23573, then starting the next cycle of treatment with AP23573.
(b) 40-80 mg of AP25373 injected every day for 5 consecutive days, followed by 2 or 9 days without additional AP23573, then starting the next cycle of treatment with AP23573.
18. Subcutaneous administration of Efalizumab in combination with oral Temsirolimus in the treatment of refractory or advanced cancers
Efalizumab is injected subcutaneously into an adult patient at a dose of 0.7 mg/kilogram of body weight, followed by weekly doses of 1 mg per kilogram (up to a maximum single dose of 200 mg), beginning two weeks prior the first administration of Temsirolimus.
Two weeks after the first Efalizumab injection, the patient begins a repeating cycle of daily dosing of Temsirolimus on one of the following schedules:
(a) 20 mg of Temsirolimus orally every day for six consecutive days, followed by 1, 8, 15 or 22 days without additional Temsirolimus, then starting the next cycle of treatment with Temsirolimus.
(b) 30-80 mg of Temsirolimus orally every day for six consecutive days, followed by 1, 8, 15 or 22 days without additional Temsirolimus, then starting the next cycle of treatment with Temsirolimus.
(c) any of the previous doses in schedule a and b, where Temsirolimus is administered orally every day for five consecutive days, followed by 2, 9, 16 or 23 days without additional Temsirolimus, then starting the next cycle of treatment with Temsirolimus.
(d) 30-75 mg of Temsirolimus orally every day for five consecutive days, every 2 weeks.
(e) 30-75 mg of Temsirolimus orally every day for 7 consecutive days, followed by 8 days without additional Temsirolimus, then starting the next cycle of treatment with Temsirolimus.
19. Subcutaneous administration of Efalizumab in combination with intravenous Temsirolimus in the treatment of refractory or advanced cancers
Efalizumab is injected subcutaneously into an adult patient at a dose of 0.7 mg/kilogram of body weight, followed by weekly doses of 1 mg per kilogram (up to a maximum single dose of 200 mg), beginning two weeks prior the first administration of Temsirolimus.
Two weeks after the first Efalizumab injection, the patient begins a repeating cycle of daily dosing of Temsirolimus on one of the following schedules:
(a) 75 mg of Temsirolimus in 30 minutes intravenous infusion on days 1, 8, 15 and 22 (i.e. day 1 being 2 weeks after the first infusion of Efalizumab). In the absence of disease progression and unacceptable toxicity, the cycle repeats every 28 days.
(b) 75-250 mg of Temsirolimus in 30 minutes intravenous infusion on days 1, 8, 15 and 22 (i.e. day 1 being 2 weeks after the first infusion of Efalizumab). In the absence of disease progression and unacceptable toxicity, the cycle repeats every 28 days.
(c) 250 mg and higher doses of Temsirolimus in 30 minutes intravenous infusion on days 1, 8, 15 and 22 (i.e. day 1 being 2 weeks after the first infusion of Efalizumab). In the absence of disease progression and unacceptable toxicity, the cycle repeats every 28 days.
20. Subcutaneous administration of Efalizumab In combination with Everolimus in the treatment of refractory or advanced cancers
Efalizumab is injected subcutaneously into an adult patient at a dose of 0.7 mg/kilogram of body weight, followed by weekly doses of 1 mg per kilogram (up to a maximum single dose of 200 mg), beginning two weeks prior the first administration of Everolimus.
Two weeks after the first Efalizumab injection, the patient begins a repeating cycle of daily dosing of everolimus on one of the following schedules:
(a) 1.5 to 5 mg of Everolimus orally every day every day for six consecutive days, followed by 1, 8, 15 or 22 days without additional Everolimus, then starting the next cycle of treatment with Everolimus.
(b) 10-20 mg of Everolimus orally every day for six consecutive days, followed by 1, 8, 15 or 22 days without additional Everolimus, then starting the next cycle of treatment with everolimus.
(c) 20 mg and higher doses of Everolimus orally every day for six consecutive days, followed by 1, 8, 15 or 22 days without additional Everolimus, then starting the next cycle of treatment with Everolimus.
(d) any of the previous doses In schedule a through c, where Everolimus is administered orally every day for five consecutive days, followed by 2, 9, 18 or 23 days without additional Everolimus, then starting the next cycle of treatment with Everolimus.
(e) any of the previous doses in schedule a through c, where Everolimus is administered orally once daily for 28 days. In the absence of disease progression and unacceptable toxicity, the treatment continues for an additional 28 days and so on.
21. Intravenous administration of Abatacept in combination with AP23573 in the treatment of refractory or advanced cancers
Abatacept is infused Intravenously over 30 minutes into an adult patient at the approved dose (see, e.g., the Table in Paragraph 0039), followed by a second and third administration two and four weeks later, respectively, and thereafter every four weeks, beginning two weeks prior the first administration of AP23573.
Two weeks after the first abatacept infusion, the patient begins a repeating cycle of daily dosing of AP23573 on one of the following schedules:
(a) 30 mg of AP23573 orally every day for six consecutive days, followed by 1, 8, 15 or 22 days without additional AP23573, then starting the next cycle of treatment with AP23573.
(b) 30 mg of AP23573 orally every day for five consecutive days, followed by 2, 9, 18 or 23 days without additional AP23573, then starting the next cycle of treatment with AP23573.
(c) 10-40 mg of AP23573 orally every day for 28 days, then starting the next cycle without interruption of treatment.
(d) 10-40 mg of AP25373 orally every day for 21 days, followed by 7 days without additional AP23573, then starting the next cycle of treatment with AP23573.
(e) 40-80 mg of AP23573 administered orally in any of the schedule a through d.
22. Intravenous administration of Abatacept in combination with intravenous AP23573 in the treatment of refractory or advanced cancers
Abatacept is infused intravenously over 30 minutes into an adult patient at the approved dose (see, e.g., the Table in Paragraph 0039), followed by a second and third administration two and four weeks later, respectively, and thereafter every four weeks, beginning two weeks prior the first administration of AP23573.
Two weeks after the first abatacept infusion, the patient begins a repeating cycle of intravenous dosing of AP23573 on one of the following schedules:
(a) 10-40 mg of AP23573 injected every day for 5 consecutive days, followed by 2 or 9 days without additional AP23573, then starting the next cycle of treatment with AP23573.
(b) 40-80 mg of AP25373 injected every day for 5 consecutive days, followed by 2 or 9 days without additional AP23573, then starting the next cycle of treatment with AP23573.
23. Intravenous administration of Abatacept in combination with oral temsirolimus in the treatment of refractory or advanced cancers
Abatacept is infused intravenously over 30 minutes into an adult patient at the approved dose (see, e.g., the Table in Paragraph 0039), followed by a second and third administration two and four weeks later, respectively, and thereafter every four weeks, beginning two weeks prior the first administration of temsirolimus.
Two weeks after the first abatacept infusion, the patient begins a repeating cycle of daily dosing of temsirolimus on one of the following schedules:
(a) 20 mg of temsirolimus orally every day for six consecutive days, followed by 1, 8, 15 or 22 days without additional temsirolimus, then starting the next cycle of treatment with temsirolimus.
(b) 30-80 mg of temsirolimus orally every day for six consecutive days, followed by 1, 8, 15 or 22 days without additional temsirolimus, then starting the next cycle of treatment with Temsirolimus.
(c) any of the previous doses in schedule a and b, where Temsirolimus is administered orally every day for five consecutive days, followed by 2, 9, 16 or 23 days without additional Temsirolimus, then starting the next cycle of treatment with Temsirolimus.
(d) 30-75 mg of Temsirolimus orally every day for five consecutive days, every 2 weeks.
(e) 30-75 mg of Temsirolimus orally every day for 7 consecutive days, followed by 8 days without additional Temsirolimus, then starting the next cycle of treatment with Temsirolimus.
24. Intravenous administration of Abatacept in combination with intravenous temsirolimus in the treatment of refractory or advanced cancers
Abatacept is infused intravenously over 30 minutes into an adult patient at the approved dose (see, e.g., the Table in Paragraph 0039), followed by a second and third administration two and four weeks later, respectively, and thereafter every four weeks, beginning two weeks prior the first administration of temsirolimus.
Two weeks after the first abatacept Infusion, the patient begins a repeating cycle of intravenous dosing of temsirolimus on one of the following schedules:
(a) 75 mg of temsirolimus in 30 minutes intravenous infusion on days 1, 8, 15 and 22 (i.e. day 1 being 2 weeks after the first infusion of Abatacept). In the absence of disease progression and unacceptable toxicity, the cycle repeats every 28 days.
(b) 75-250 mg of temsirolimus in 30 minutes intravenous infusion on days 1, 8, 15 and 22 (i.e. day 1 being 2 weeks after the first infusion of Abatacept). In the absence of disease progression and unacceptable toxicity, the cycle repeats every 28 days.
(c) 250 mg and higher doses of temsirolimus in 30 minutes intravenous infusion on days 1, 8, 15 and 22 (i.e. day 1 being 2 weeks after the first infusion of Abatacept). In the absence of disease progression and unacceptable toxicity, the cycle repeats every 28 days.
25. Intravenous administration of Abatacept in combination with Everolimus in the treatment of refractory or advanced cancers
Abatacept is infused intravenously over 30 minutes into an adult patient at the approved dose (see, e.g., the Table in Paragraph 0039), followed by a second and third administration two and four weeks later, respectively, and thereafter every four weeks, beginning two weeks prior the first administration of Everolimus.
Two weeks after the first Abatacept infusion, the patient begins a repeating cycle of daily dosing of Everolimus on one of the following schedules:
(a) 1.5 to 5 mg of Everolimus orally every day every day for five or six consecutive days, followed by 1, 8, 15 or 22 days without additional Everolimus, then starting the next cycle of treatment with Everolimus.
(b) 10-20 mg of Everolimus orally every day for six consecutive days, followed by 1, 8, 15 or 22 days without additional Everolimus, then starting the next cycle of treatment with Everolimus.
(c) 20 mg and higher doses of Everolimus orally every day for six consecutive days, followed by 1, 8, 15 or 22 days without additional Everolimus, then starting the next cycle of treatment with Everolimus.
(d) any of the previous doses in schedule a through c, where Everolimus is administered orally every day for five consecutive days, followed by 2, 9, 16 or 23 days without additional Everolimus, then starting the next cycle of treatment with Everolimus.
(e) any of the previous doses in schedule a through c, where Everolimus is administered orally once daily for 28 days. In the absence of disease progression and unacceptable toxicity, the treatment continues for an additional 28 days and so on.
26. Examples 1-25 may be practiced using golimumab, tocilizumab, certolizumab pegol or anakinra, at their respectively approved doses, routes of administration and schedules as noted previously, in place of infliximab, etanercept, Humira, efalizumab or abatacept, respectively.

1. A method comprising the steps of administering to a cancer patient both
(a) a first drug selected from AP23573, temsirolimus and everolimus; and
(b) a second drug selected Remicade, Humira, Enbrel, Raptiva, Orencia, Golimumab, Actemra, Cimzia and Kineret.

2. The method according to claim 1, wherein administration of the second drug is initiated prior to initiation of administration of the first drug.

3. The method according to claim 2, wherein administration of the second drug is initiated at least one day prior to the first administration of the first drug.

4. The method of claim 2, wherein administration of the second drug is initiated 7-21 days prior to the first administration of the first drug.

5. The method of claim 1, wherein the first drug is administered orally.

6. The method of claim 1, wherein the first drug is administered by intravenous infusion.

7. The method of claim 5, wherein the second drug is administered subcutaneously.

8. The method of claim 5, wherein the second drug is administered intravenously.

9. The method of claim 6, wherein the second drug is administered subcutaneously.

10. The method of claim 6, wherein the second drug is administered intravenously.

11. The method of claim 1, wherein the first drug is administered at an average weekly dose level of 25 to 400 mg.

12. The method of claim 1, wherein the first drug is administered at an average weekly dose level of 100 to 500 mg.

13. The method of claim 1, wherein the cancer is a sarcoma, lymphoma, or leukemia or a cancer of the bladder, colon, brain, breast, head and neck, endometrium, lung, ovary, pancreas or prostate.
